(19) United States
(12) Patent Application Publication
(10) Pub. No.: US 2025/0313793 A1
(43) Pub. Date: Oct. 9, 2025

(54) AUTOMATED CELL CULTURING AND CHARACTERIZATION TO RESEMBLE IN VIVO CONDITIONS

(71) Applicant: University of Virginia Patent Foundation, Charlottesville, VA (US)

(72) Inventors: Robin A. Felder, Charlottesville, VA (US); John J. Gildea, Waynesboro, VA (US); Peng Xu, Charlottesville, VA (US); Mathew Chase Carson, Charlottesville, VA (US)

(21) Appl. No.: 18/873,163

(22) PCT Filed: Jun. 9, 2023
(86) PCT No.: PCT/US2023/068211

§ 371 Date: Dec. 9, 2024

Related U.S. Application Data
(60) Provisional application No. 63/350,677, filed on Jun. 9, 2022.

(51) Int. Cl. C12M 1/36; C12M 1/00; C12M 1/12; C12M 1/26; C12M 1/34; C12M 3/04; C12N 5/00

(52) U.S. Cl. CPC C12M 41/48; C12M 27/10; C12M 29/06; C12M 33/04; C12M 37/00; C12M 41/26; C12M 41/46; C12N 5/0075; C12N 2533/30

(57) ABSTRACT

A method of automated cell culturing and characterization can include regulating one or more parameters of an ambient environment within an environmentally isolated, airtight enclosure. A biological specimen can be cultured within the enclosure, e.g., including aspirating and dispensing, via an automated fluid handling system having a fluidic interface disposed within the airtight enclosure, a portion of the biological specimen. The dispensing can be into a first vessel, e.g., by suspending individual cells of the portion of the biological specimen within a microcarrier matrix contained by the first vessel and exposed to the ambient environment within the airtight enclosure.

### CLAIM OF PRIORITY

This application claims priority to U.S. Provisional Application Ser. No. 63/650,677, filed on Jun. 9, 2022, which is incorporated by reference herein in its entirety, and the benefit of priority of which is claimed herein.

### STATEMENT REGARDING FEDERALLY SPONSORED RESEARCH OR DEVELOPMENT

This invention was made with government support under Grant No. HL074940 awarded by the National Institutes of Health. The government has certain rights in the invention.

### BACKGROUND

Cell culturing can be used in certain restorative and regenerative medicine procedures and for therapeutic treatments for a myriad of human diseases, such as cancer and diabetes. Furthermore, cell culturing can be used to study physiological behavior, metabolism, development of diseases, and the effects of various drugs and therapies. The viability of an individual cell culture varies based on the culture environment, e.g., the environment's ability to provide, nutrients, oxygen, stable temperature, and a balanced pH. Generally, cell culturing can involve specialized stand-alone equipment, e.g., a cell culture hood, cell culture incubator, cell culture shaker, pipettor, and microscope, as well as specific reagents and media. Generally such culturing equipment is operated manually and involves significant human interaction with each unit operation. For example, cultures can be monitored and evaluated by skilled personnel for signs of cell stress, growth rate, and maturation. A wide variety of cell types can be used in an attempt to grow a viable cell culture, e.g., as primary cells, stem cells, or progenitor cells.

### SUMMARY

One approach to cell culturing in a lab can involve a two-dimensional (2D) substrate, such as petri dishes or plastic flasks, e.g., in a liquid nutrient-rich media. Such approaches can be challenging or limiting regarding the scale and complexity of the cell cultures that can be grown with requisite success. Another approach to cell culturing in a lab can involve one or more stand-alone bioreactors wherein three-dimensional (3D) structures can be provided, e.g., to imitate an environment of living tissue more closely in the body, thereby enabling culturing of more complex and lifelike cell cultures. One challenge involved in cell culturing using certain bioreactors is that it can be difficult to obtain a desired level of control of certain parameters of the environment, such as temperature and pH. Additionally, certain bioreactors can be relatively large and expensive pieces of equipment, which can be difficult to monitor and culture cells to a desired scale. Furthermore, the purity of the culture environment in certain bioreactors can involve costly and time-consuming sterilization processes. The present inventors have recognized a need for an automated system for cell culturing in bioreactors including 3D structures, e.g., including an isolated environment that does not require manual interaction or disruption of parameters of the environment during inoculation or monitoring of the growing cell cultures.

This document describes a method of automated cell culturing and characterization, e.g., to resemble certain in vivo environment conditions and promote desired cellular growth. Such a method can involve regulating one or more parameters of an ambient environment within an environmentally isolated, airtight enclosure. A first biological specimen variety can be received and cultured within the airtight enclosure. For example, the ambient environment within the airtight enclosure can remain environmentally isolated from an outside environment during the culturing of the first biological specimen variety.

Culturing of the first biological specimen can include aspirating, via an automated fluid handling system having a fluidic interface disposed within the airtight enclosure, a portion of the first biological specimen variety. Such culturing can also include, dispensing, via the fluidic interface of the automated fluid handling system, the portion of the first biological specimen variety in a first vessel, e.g., including suspending individual cells of the portion of the first biological specimen variety within a microcarrier matrix contained by the first vessel and exposed to the ambient environment within the airtight enclosure. At least one cellular growth indicator can be monitored within the first vessel over time.

Mechanical movement of the first vessel can be established or adjusted, such as based on the at least one cellular growth indicator, to promote growth of an ex vivo cell culture within the first vessel. For example, establishing or adjusting the mechanical movement of the first vessel can include bidirectionally oscillating the first vessel via alternating a rotation of the vessel at least 180° in each direction. Also, a rotational motion from the bidirectional oscillation can be translated to lateral, vertical motion of the microcarrier matrix contained by the first vessel.

In an example, culturing the first biological specimen variety can include controlling movement, via a robotic manipulator, of the fluidic interface of the automated fluid handling system, toward the first vessel. In an example, controlling the movement of the fluidic interface can include placing a pipette tip of the fluidic interface within ±0.3 mm of a target location. Culturing the first biological specimen variety can also include monitoring a pH value within the first vessel over a specified duration, e.g., changing fluid within the first vessel upon a determination that a change in pH exceeds a specified threshold.

In an example, the method can include receiving a second biological specimen variety within the airtight enclosure. Here, the second biological specimen variety can be cultured similar to the first biological specimen variety, including aspirating, via the automated fluid handling system disposed within the airtight enclosure, a portion of the second biological specimen variety and dispensing the portion of the second biological specimen variety within a second vessel. For example, individual cells of the portion of the second biological specimen variety can be suspended within a microcarrier matrix contained by the first vessel and exposed to the ambient environment within the airtight enclosure. In an example, at least one cellular growth indicator can be monitored within the second vessel over time. In an example, an oscillation of the second vessel can be established, e.g., based on the at least one cellular growth indicator, to promote growth of a cell culture within the second vessel. Where a plurality of different biological specimen varieties are cultured within the airtight enclosure, a pipette tip of a fluidic interface included in the fluid handling system can be sterilized or washed between handling of the different biological specimen varieties. For example, the pipette tip can be sterilized or washed between the dispensing of the portion of the first biological specimen variety within the first vessel and the aspirating the portion of the second biological specimen variety. For example, the washing or sterilizing the pipette tip can include moving the fluidic interface, via a robotic manipulator, toward a washing or sterilization unit disposed within the environmentally isolated, airtight enclosure.

Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The detailed description is included to provide further information.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various examples discussed in the present document.

FIG. 1A is a perspective view of an example of an automated cell cultivation system including an environmentally isolated, airtight enclosure.

FIG. 1B is an isometric view showing inside an airtight enclosure of an automated cell cultivation system.

FIG. 1C is a detailed view showing an inside of an airtight enclosure of an automated cell cultivation system.

FIG. 2A is a perspective view of an isolated example of a fluid handling system, including a plurality of bioreactor vessels.

FIG. 2B depicts exemplary arrangements of bioreactor vessels, including a detailed view of an individual bioreactor vessel.

FIG. 3A is a perspective view of an isolated example of a robotic manipulator arranged to control movement of a fluidic interface an exemplary automated fluid handling system.

FIG. 3B is a detailed view of a robotic manipulator of an exemplary automated fluid handling system.

FIG. 4 depicts a user interface for monitoring of an example of an automated cell cultivation system.

FIG. 5 is a plot depicting concurrent monitoring of a plurality of different exemplary bioreactor vessels.

FIG. 6 is a flowchart that describes a method of automated cell culturing and characterization.

FIG. 7 is a block diagram illustrating components of a machine.

### DETAILED DESCRIPTION

Certain approaches to automated cell culturing focus generally on monolayer cultures, e.g., anchorage-dependent cells grown on a two-dimensional (2D) substrate such as a Petri dish, culture plate, flask, or other vessel promoting cell growth primarily on walls or surfaces thereof. A challenge with culturing approaches using 2D substrates is they can provide an environment providing significantly different conditions than those observed in vivo.

For example, certain cancer cells grow relatively rapidly in culture, and can generally be grown to a viable culture using a 2D substrate. This is related to the fact certain cancer cells can generally grow in environments where cells can struggle, e.g., on plastic or glass. Also, these certain cancer cells can withstand oxygen tension from about 21% to near zero between culturing, as well as withstand extreme glucose, lactate, glutamine, and ammonia concentrations as compared to in vivo conditions. A particular challenge with studying anchorage-dependent cancer cell cultures grown on 2D substrates is that certain cancer cells of interest, e.g., metastatic cells with stem cell characteristics, are generally far less resilient than other certain cancer cells that are relatively rapid-growing, which can complicate isolating such cancer cells of interest in culture. Additionally, cancer cells grown on 2D substrates generally grow as flat sheets, exhibiting relatively high integrin engagement with surrounding cells. This growth behavior is significantly different from that observed in primary cancer cells in vivo, involving disorganized cell-to-cell interactions exhibiting relatively low integrin engagement with surrounding cells.

Further, while endothelial cells and fibroblasts can generally be grown in 2D substrates, certain cell types do not grow well as anchorage-dependent cell cultures. For example, epithelial cells include, in vivo, a cellular architecture for establishing a separation or a barrier, e.g., via polarization, tight junctions, adherent junctions, or primary cilia exhibiting polarity, differing electrical potential, and polar or differential solute absorption. As such, the growth of epithelial cells in vivo generally involves a requisite stiffness and composition of a basement layer containing different combinations of, e.g., collagen, elastin, fibronectin, laminin, vitronectin, and hyaluronic acid such that growth factor can be presented to the epithelial cells as they occur in the body, e.g., through transient interaction with the basement layer delivered from the basolateral side of the cell. Such conditions can be difficult to resemble in anchorage-dependent cell cultures involving 2D substrates.

In another approach, a three-dimensional (3D), anchorage-independent cell culture can be grown. Certain 3D cell culture methods can involve growth substrates and shapes that more closely resemble those in a mammalian body and can better facilitate an investigation of the cell behavior in constructs similar to in vivo conditions. Such 3D surfaces can support the natural hypoxic conditions and development of extracellular matrix similar to in vivo microenvironments. For example, certain scaffold-based culture systems can include a carrier material such as to support cell growth in 3D environments. This can be facilitated, e.g., by seeding the cells onto acellular matrices or by encapsulating the cells in gels and polymerizing the substrate.

A challenge with such approaches involving 3D, anchorage-independent cell cultures is these approaches generally involve manual methods that are labor-intensive and challenging to control. For example, contamination of these anchorage-independent cell cultures, e.g., by bacteria or viruses, can result in reduced cell growth and can ultimately lead to cell death. Such contaminants can be ubiquitous and can be relatively easily transmitted from culture to culture. The present inventors have recognized a need for an automated technique for culturing within a sterile environment with minimal human contact and accommodating culturing of 3D, anchorage-independent cell cultures as well as 2D, anchorage-dependent cell cultures.

This document describes an automated cell cultivation system capable of supporting 3D cell culture methods to grow and maintain living cells. The system can help enable a desired bandwidth and consistency in cell culturing, e.g., by limiting certain variances generally introduced by human error, e.g., by providing a sterile enclosure in which a robotic manipulator performs cell culturing steps and limiting outside interaction from a cell culture technician. In an example, the automated cell cultivation system can accommodate about 64 different cell lines being cultivated concurrently or in a random-access sequence, e.g., in individually controllable bioreactors.

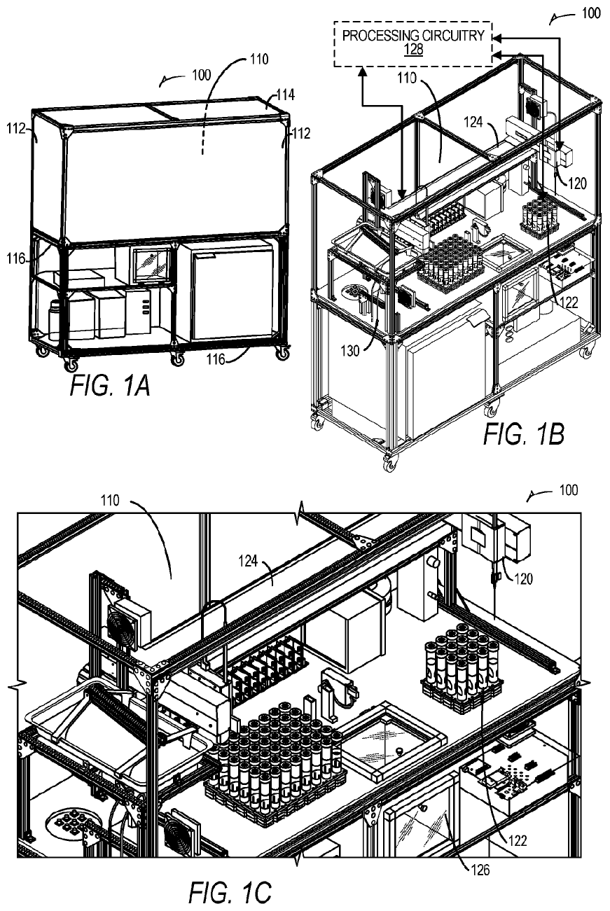

FIG. 1A, FIG. 1B, and FIG. 1C depict an example of an automated cell cultivation system. The system 100 can include an environmentally isolated, airtight enclosure 110 for defining an ambient environment therewithin. The enclosure 110 can limit fluid communication with an outside environment. In an example, as depicted in FIG. 1A, the enclosure 110 can be defined by walls 112, a top 114, and a base 116. In an example, at least one of the walls 112, top 114, or base can be formed of a non-opaque material, e.g., transparent polycarbonate, to enable visibility into the enclosure 110 without introducing fluid communication with the outside environment. In an example, air filtration, such as HEPA filtration, can facilitate the enclosure 110 maintaining a sterile environment. In an example, the enclosure 110 can limit the introduction of foreign contaminants, or allow the introduction of desired nutrients, e.g., downstream of an air filtration unit. This can help prevent spores, bacterial, or viral contaminants from entering the enclosure of the system 100. In an example, passage areas, corners, and edges around the enclosure 110 can reinforced, e.g., with aluminum tape to help promote airtight sealing.

The system can be mounted on a frame 118, e.g., formed of anodized extruded aluminum, and a plurality of casters can also be mounted to the frame. One or more auxiliary apparatus, e.g., a refrigerator, a storage reservoir, a specimen reheater, a control system, or a power system, can be mounted to the frame 118 and can be included in or used by the system 100.

FIG. 1B and FIG. 1C show visibility into an airtight enclosure of the automated cell cultivation system 100, the walls 112 and the top 114 having been omitted in the depictions. In an example, the automated cell cultivation system 100 can include a fluid handling system 120, a plurality of specimen vessels 122, a robotic manipulator 124, an air lock chamber 126, and processing circuitry 128. In an example, any one of the fluid handling system (or a fluidic interface thereof), the plurality of specimen vessels 122, the robotic manipulator 124, and the air lock chamber 126, can be fully enclosed within the enclosure. The system 100 can also include one or more regulation systems, e.g., a humidity regulation system (e.g., including a water evaporation pan), a temperature regulation system (including, e.g., a heater), and a gas (e.g., O₂ or CO₂) regulation system.

The air lock chamber 126 can include a sterilizing device, such as, e.g., one or more ultraviolet (UV) lights, one or more heating devices, one or more pressure or vacuum cycles, and/or one or more chemical agents or treatments to facilitate sterilization of objects, fluids, or gases entering or exiting the chamber 126. The air lock chamber 126 can be configured in any appropriate manner, e.g., to limit the introduction of contaminants into the enclosure 110 while generally maintaining a desired airtight environment as described herein. In an example, the air lock chamber 126 can be accessible such as to replace individual specimen vessels 122 within the enclosure 110 while limiting a change in internal environment parameters (e.g., temperature, gas concentration, humidity, etc.) during the using of the air lock chamber 126. In an example, the system 100 can also include one or more portholes including glovebox-style gloves to load or remove individual specimen vessels 122 within the enclosure 110, e.g., without accessing the air lock chamber 126.

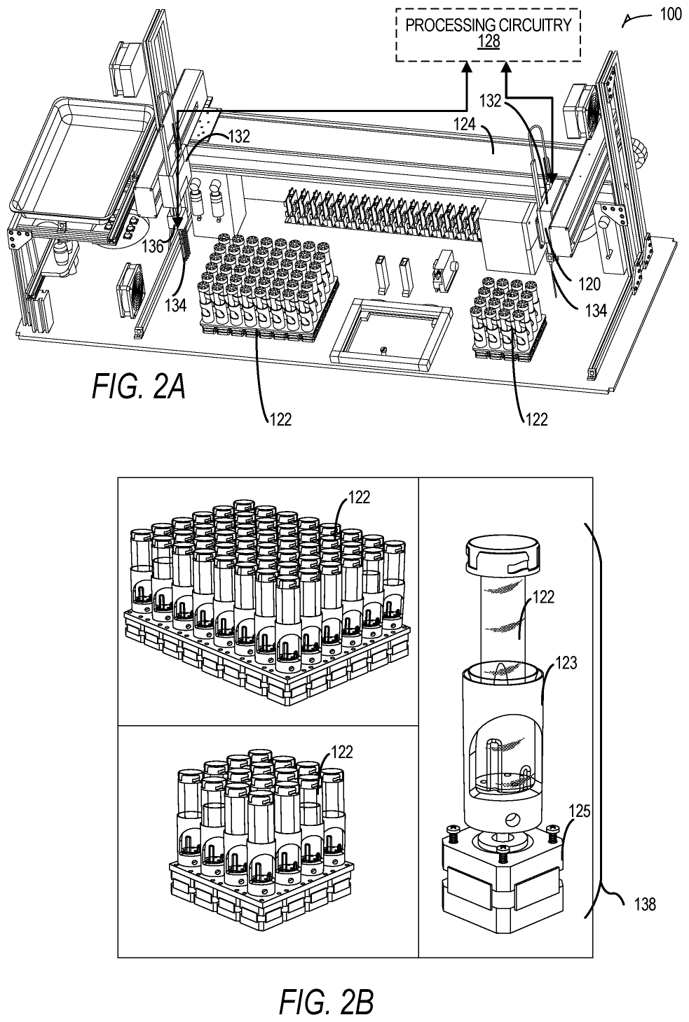

The processing circuitry 128 can be communicatively coupled with any one of the fluid handling system 120, the specimen vessels 122 or corresponding vessel receptacles (e.g., receptacle 123 as depicted in FIG. 2B), the robotic manipulator 124, the air lock chamber 126, and any of the one or more regulation systems. In an example, the processing circuitry 128 can control a respective one or more regulation systems to regulate corresponding internal environmental parameters of an airtight environment within the enclosure 110 according to ranges or default settings within the below table.

TABLE 1
Illustrative (but non-limiting) examples of
environmental parameters that can be used.

| | | Default | |
|---|---|---|---|
| Parameter | Range | Settings | Sensor |
| O₂ concentration [%] | 10-21 | 20 ± 1 | O2-BTA |
| CO₂ concentration [%] | 0-10 | 5 ± 0.5 | CO2- |
| | | | BTA |
| Temperature [° C.] | 10-40 | 37 ± 1 | SHT15 |
| Temperature [° F.] | 50-104 | 98.6 ± 1.8 | |
| Relative humidity | 30%-80% (depending | >50% ± 5% | |
| | on ambient condition | | |
| | in room) | | |
| pH value of cell | 0-14 | 7.4 ± 0.01 | WASFET |
| culture medium | | | |

In an example, the processing circuitry 128 can control the temperature regulation system to regulate an ambient temperature inside the enclosure 110 within a range between about 35° F. and about 140° F. The processing circuitry 128 can control the humidity regulation system to regulate an ambient temperature inside the enclosure 110 within a range between about 30% RH and about 100% RH. In an example where the processing circuitry 128 is configured to control regulation of the relative humidity toward 100% RH, the system 100 can activate one or more heating units to help warm certain humidity-sensitive components (e.g., processors, gas sensors, etc.) to help avoid any challenges arising from condensation within the enclosure 110. In an example, the processing circuitry 128 can control the gas regulation system (either of CO2 or O2) to regulate a respective gas concentration inside the enclosure 110 within a range between 0% and about 100%. In an example, the processing circuitry 128 can also control the gas regulation system to release nitrogen (N2) within the enclosure 110, e.g., as purge gas.

Each of the one or more regulation systems can include or use one or more corresponding sensor 130, e.g., a relative humidity sensor, a thermostat, a gas sensor, etc. For example, a relative humidity and temperature can be determined, e.g., using a SHT15 sensor (Sensirion, Stäfa, Switzerland), which can be mounted, e.g., on a horizontal linear rail of robotic manipulator. Where the sensor 130 is a temperature sensor, the sensor 130 can determine a temperature within a range of about −40° C. to about 123.8° C. (about −40° F. to about 254.9° F.), at an accuracy of about ±0.3° C. (about ±0.54° F.), and a repeatability of about ±0.1° C. (about ±0.18° F.). Where the sensor 130 is a relative humidity (RH) sensor, the sensor 130 can determine an RH within a range of about 0% to about 100% RH, at an accuracy of about ±2.0% RH, and at a repeatability of about ±0.1% RH.

An example of a sensor 130 for air composition measurement for use in a corresponding regulation system is an oxygen sensor O²-BTA or a carbon dioxide sensor CO²-BTA from Vernier Software & Technology (Beaverton, OR, USA). Where the sensor 130 is an oxygen (O²) sensor, the sensor 130 can determine an O² concentration within a range of about 0% to about 27%, at an accuracy of about ±1%, and at a resolution of about ±0.01%. Where the sensor 130 is a carbon dioxide (CO²) sensor, the sensor 130 can determine a CO² concentration within a range from about 0% to about 10% at an accuracy of about ±10% and at a resolution of about ±0.0012%. In an example, environmental conditions within the enclosure 110 can be regulated by a proportional-integral-derivative (PID) controller system. One or more pneumatic valves can be attached to respective gas supply tanks (e.g., O², CO², or N²) to help control an airflow from a corresponding tank into the enclosure 110. In an example, the system 100 can include one or more circulating fans to assist with rapid change of state and mixing of the supplied gases. In an example for temperature control, a plurality of heaters can be attached to a circulating fan, and the heater circuit can be powered by a solid-state relay.

FIG. 2A is a perspective view of an isolated example of a fluid handling system, including a plurality of bioreactor vessels. A fluid handling system 120 can include one or more fluidic interfaces 132 disposed within the airtight enclosure. An individual fluidic interface 132 can include one or more pipette tips 134, e.g., including a syringe. In an example, the fluid handling system can be arranged, e.g., by operation of the processing circuitry 128, to aspirate a portion of a biological specimen variety through an individual pipette tip 134. In an example, prior to aspiration biological specimen variety can be disposed in, e.g., a reservoir within the enclosure 110 (enclosure depicted in FIG. 1B), a container, or via piping or tubing from a reservoir disposed outside the enclosure 110. The fluid handling system can also be arranged to dispense, e.g., out the pipette tip 134, the portion of aspirated the biological specimen variety in a specimen vessel 122, e.g., a different specimen vessel 122 than a vessel from which it was aspirated. Here, the fluid handling system can facilitate transfer of the biological specimen variety, cell culture media, or both from one container (e.g., a vessel 122) to another. In an example, the biological specimen variety, cell culture media, or both can be piped through a re-heater (e.g., at about 37° C.) to avoid shocking the cells during media delivery. The ambient environment within the airtight enclosure 110 can remain environmentally isolated from an outside environment during the selective aspiration and dispensing of the biological specimen variety.

In an example, the system 100 can include at least one pH sensor 136 for measuring a pH value within the vessel 122 over a specified duration. For example, pH sensor 136 can be communicatively coupled with the processing circuitry 128. Here, the processing circuitry 128 can determine, via data received from the pH sensor 136, a change in pH over the specified duration exceeding a specified threshold, and the specified threshold can be predetermined based on cellular attributes of the biological specimen variety. The specified threshold can be a threshold variance from a target value, e.g., greater than ±0.01 variance from a target pH of 7.1. In response to a determination that a change in pH over the specified duration exceeds the specified threshold, the processing circuitry 128 can trigger replacement of fluid within the vessel 122, e.g., via the fluid handling system 120. In an example, the pH sensor 136 can include a WASFET pH-sensor kit (e.g., from Sentron Europe BV (Leek, Netherlands)). The pH sensor 136 can determine a pH within a range from about pH 0.00 to about pH 14.00 and at an accuracy of ±0.01. In an example, the fluid handling system 120 can transport liquid medium samples to a pH-sensing station and the processing circuitry 128 can monitor the change in pH to determine the correct time for media change, of an individual specimen vessel 122. After the measurement, the pH-sensing station can be flushed with water or a disinfecting agent to avoid cross-contamination.

In an example, an individual fluidic interface 132 can include one or more (e.g., four) PTFE coated steel pipette tips 134. Where the individual fluidic interface 132 includes more than one pipette tip 134, each individual pipette tip 134 can be arranged to deliver a different cell media from another individual pipette tip 134. In an example, one or more pipette tips 134 can be driven by a syringe pump (e.g., an XLP 6000 pump from Tecan), establishing, e.g., a syringe volume within a range of about 1 mL and about 50 mL and defining a multiport valve. In an example, an individual fluidic interface 132 can be connected to an ethanol (EtOH) storage reservoir or a water storage reservoir. The fluid handling system 120 can include an individual fluidic interface 132 connected via a network (e.g., Ethernet) to the central computer and control software. In an example, the fluid handling system 120 can communicate via Controller Area Network (CAN) with the robotic manipulator 124 and with each syringe pump controller of an individual fluidic interface 132.

FIG. 2B depicts exemplary arrangements of bioreactor vessels, including a detailed view of an individual bioreactor vessel. In an example, the system 100 can include an individual vessel 122. The individual vessel 122 can also include a microcarrier matrix contained therein. The microcarrier matrix can be arranged for receiving individual cells of the portion of the biological specimen variety. In an example, the microcarrier matrix can include a ferromagnetic-infused biomimetic hydrogel microcarrier matrix included such as to receive the individual cells of the biological specimen and suspend the individual cells throughout the hydrogel microcarrier matrix during the culturing of the biological specimen variety. The biological specimen variety can include an animal cell, a cell line, a plant cell, a micro-organism, a tissue or an organ, among other varieties.

Microcarrier suspension of cell from the biological specimen variety can be achieved with one or more bioreactors 138 including an oscillator 125 (e.g., a Wiggler™) arranged to provide bidirectional ˜360° rotations around a central axis, e.g., under computer programmed speed control (e.g., Wiggling™). An individual bioreactor 138 can also include a vessel receptacle 123 e.g., attached to a drive shaft of the oscillator 125. The specimen vessels 122 (e.g., Levitubes™) can include interior fins arranged transform rotational motion of the tube into a gentle upward lifting, such as facilitate even suspension of microcarriers (e.g., global eukaryotes microcarriers (GEMs™)) in the cell culture media. Speed of the bidirectional rotation, which can be defined as wiggles per minute (WPM), can be determined such as to avoid a tendency of microcarriers to develop groups of attached microcarriers (e.g., clumps). In an example, faster rotation can limit clumping of the GEMs™ in cell lines. In an example, an individual bioreactor 138 can maintain cell populations of up to about 2 billion cells e.g., in the form of suspended organoids for a relatively long period (e.g., up to several months).

In an example, the processing circuitry 128 can be connected to at least one individual bioreactor 138 and configured to monitor at least one cellular growth indicator within the individual vessel 122 over time. For example, the at least one cellular growth indicator can include at least one of size, density of the portion of the biological specimen variety, quality of cellular features, number of secreted proteins, etc. Here, the processing circuitry 128 can establish or adjust mechanical movement of the vessel 122, based on the at least one cellular growth indicator, e.g., to promote growth of an ex vivo cell culture within the vessel 122.

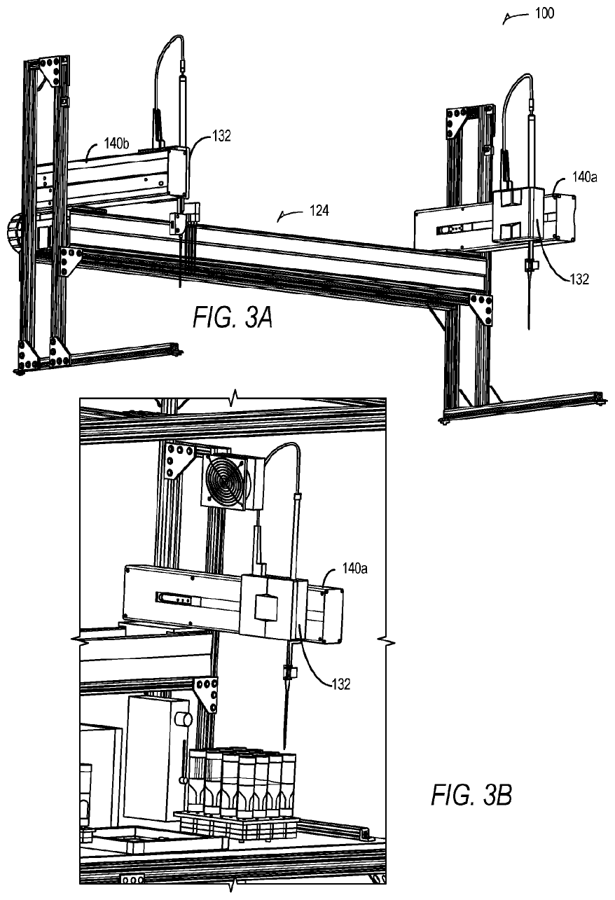

FIG. 3A and FIG. 3B depict an example of a robotic manipulator arranged to control movement of a fluidic interface an exemplary automated fluid handling system. In an example, the system 100 can include a robotic manipulator 124 (e.g., a Cavro® Omni Robot, Tecan, Morrisville, NC, USA)) including a first arm 140a and a second arm 140b. Here each of the arms 140a and 140b can be coupled with an individual fluidic interface 132 and arranged to move a plurality of fluidic interfaces 132 independent of one another. An individual arm 140 can be moveable along at least three axes, e.g., along tracks or within grooves of a manipulator frame, to position the individual fluidic interface 132. The robotic manipulator 124 can also be operated, e.g., by the processing circuitry 128 (as depicted in FIG. 1B) to move an individual fluidic interface 312 toward the washing or sterilization unit disposed within the enclosure 110 (as depicted in FIG. 1B), e.g., to facilitate at least one of washing or sterilizing a pipette tip of a fluidic interface included in the fluid handling system 120. In an example, each arm 140a and 140b of the robotic manipulator 124 can cover a working area of about 125.0×about 30.0×about 21.0 cm (about 49.2×about 11.8×about 8.3 inches). Each arm 140a and 140b can be capable of lifting a maximum payload of about 6.6 kg with an accuracy of about ±0.3 mm and repeatability lower than about 0.2 mm. In an example, the robotic manipulator can move the individual fluidic interfaces 132 while working within an operating temperature range of about 10° C. to about 40° C. (about 50° F. to about 104° F.) and relative humidity range from about 30% up to about 80%.

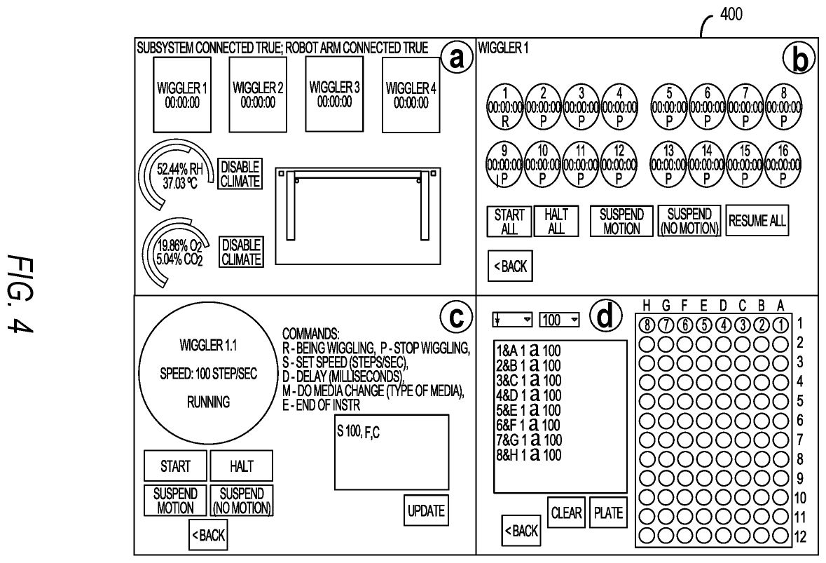

FIG. 4 depicts a user interface for monitoring of an example of an automated cell cultivation system. In an example, cell culturing in an automated cell cultivation system can involve remote monitoring, e.g., using a user interface or a web application 400. The user interface 400 can display status parameters and sensor values corresponding with a particular bioreactor. In an example, the user interface 400 can be updated at a specified frequency (e.g., every 5 seconds) and can include a video stream for an insight into certain system operations and cellular growth within a bioreactor. As depicted in FIG. 4, control software with graphical user interface can include—(a) a main view with status of bioreactor (wiggler) arrays, systems climate, and atmosphere, as well as the liquid handlers arm positions; (b) a view of bioreactor (Wiggler™) array 1, status and command fields; (c) view of individual bioreactor (Wiggler™) 1.1 status and command fields; and (d) view of a microplate and command fields. In an example, the main view a of the user interface 400 can display an overview of the status of the bioreactor groups, e.g., the system's climate and atmosphere, as well as the robotic manipulator arm coordinates or positions. The user interface 400 can include inputs to control actions of the system processor 128 (as depicted in FIG. 1B) such as commands to start or stop oscillation (wiggling), suspend action (with or without continued motion) or resume any action (e.g., if previously stopped manually or by system error). The user interface 400 can also include inputs to control actions of the system processor 128 such as commands to specify an oscillation rate (wiggles per minute, exposed as steps/second) to adjust shear force, include a delay time (milliseconds), or to execute a media change with a selected media type.

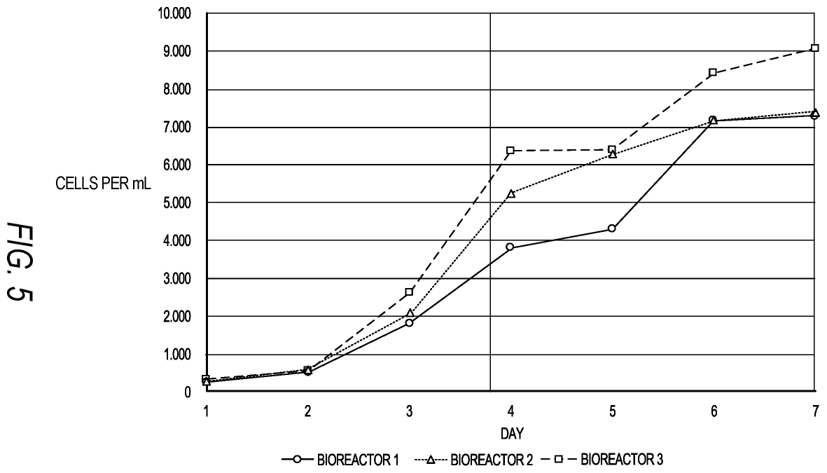

FIG. 5 is a plot depicting the concurrent monitoring of a plurality of different exemplary bioreactor vessels. A robotic system can facilitate a plurality of concurrently grown 3D cell cultures, e.g., to produce large quantities of cells. As depicted from the exemplary data in FIG. 5, the automated cell cultivation system can help regulate stable environmental conditions for growth of a plurality of cultures. In an example, the system can concurrently grow and maintain about 64 individual cell cultures using 3D suspension via oscillation of the cells growing within the microcarrier matrix.

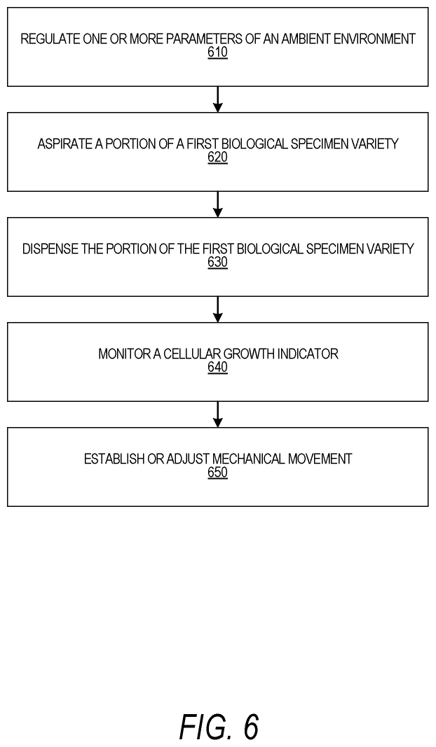

FIG. 6 is a flowchart that describes a method of automated cell culturing and characterization.

In an example, at 610, the method can include regulating one or more parameters of an ambient environment within an environmentally isolated, airtight enclosure. For example, a first biological specimen variety can be received within the airtight enclosure. For example, the one or more parameters of the ambient environment within the airtight enclosure can be established or adjusted to maintain an ambient temperature within a range from 50° Fahrenheit (F) to 150° F., or within a range from 93° Fahrenheit (F) to 107° F. The one or more parameters of the ambient environment within the airtight enclosure can be established or adjusted to maintain a relative humidity (RH) within a range from 75%-100%, or within a range of 45%-80%. The one or more parameters of the ambient environment within the airtight enclosure can be established or adjusted to maintain a CO² concentration within a range from 0%-15%. The one or more parameters of the ambient environment within the airtight enclosure can be established or adjusted to maintain a O² concentration within a range from 5%-25%.

Generally, the method can involve culturing a first biological specimen variety. For example, at 620, the culturing can include aspirating, via an automated fluid handling system having a fluidic interface disposed within the airtight enclosure, a portion of the first biological specimen variety. The ambient environment within the airtight enclosure can remain environmentally isolated from an outside environment during the culturing of the first biological specimen variety.

At 630, the culturing can include dispensing, via the fluidic interface of the automated fluid handling system, the portion of the first biological specimen variety in a first vessel, including suspending individual cells of the portion of the first biological specimen variety within a microcarrier matrix contained by the first vessel and exposed to the ambient environment within the airtight enclosure.

At 640, the culturing can include monitoring at least one cellular growth indicator within the first vessel over time, e.g., at a location outside the airtight enclosure.

At 650, the culturing can include establishing or adjusting mechanical movement of the first vessel, based on the at least one cellular growth indicator, to promote growth of an ex Vivo cell culture within the first vessel.

In an example, culturing the first biological specimen variety can also controlling movement, via a robotic manipulator, of the fluidic interface of the automated fluid handling system, toward the first vessel. For example, a pipette tip of the fluidic interface can be placed via the robotic manipulator within ±0.3 mm of a target location.

The method can also include disposing a ferromagnetic-infused biomimetic hydrogel microcarrier matrix within the first vessel, the hydrogel microcarrier matrix arranged to receive the individual cells of the biological specimen and suspend the individual cells throughout the hydrogel microcarrier matrix during the culturing of the first biological specimen variety.

In an example, a second biological specimen variety can be received within the airtight enclosure. The second biological specimen variety can be aspirated and dispensed similar to the first biological specimen variety, including suspending individual cells of the portion of the second biological specimen variety within a microcarrier matrix contained by a second vessel and exposed to the ambient environment within the airtight enclosure. At least one cellular growth indicator can be monitored within the second vessel over time. Also, an oscillation of the second vessel can be established or adjusted, based on the at least one cellular growth indicator, to promote growth of a cell culture within the second vessel. A pipette tip of a fluidic interface included in the fluid handling system can be washed or sterilized between the dispensing of the portion of the first biological specimen variety within the first vessel and the aspirating the portion of the second biological specimen variety. For example, washing or sterilizing the pipette tip can include moving the fluidic interface, via a robotic manipulator, toward a washing or sterilization unit disposed within the environmentally isolated, airtight enclosure.

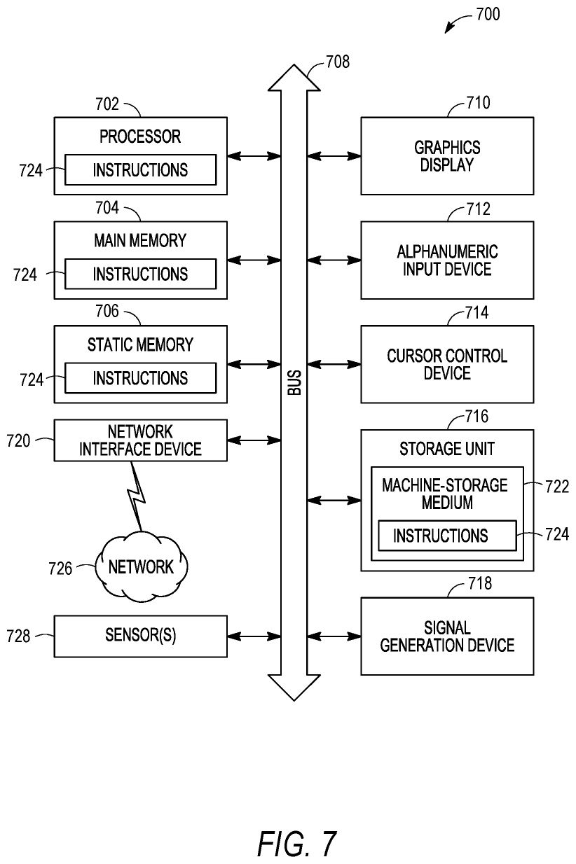

FIG. 7 is a block diagram illustrating components of a machine 700, according to some example embodiments, able to read instructions 724 from a machine-storage medium 722 (e.g., a non-transitory machine-storage medium, a machine-storage medium, a computer-storage medium, or any suitable combination thereof) and perform any one or more of the methodologies discussed herein, in whole or in part. Specifically, FIG. 7 shows the machine 700 in the example form of a computer system (e.g., a computer) within which the instructions 724 (e.g., software, a program, an application, an applet, an app, or other executable code) for causing the machine 700 to perform any one or more of the methodologies discussed herein can be executed, in whole or in part. For example, the instructions 724 can be processor executable instructions that, when executed by a processor of the machine 700, cause the machine 700 to perform the operations outlined above.

In various embodiments, the machine 700 operates as a standalone device or can be communicatively coupled (e.g., networked) to other machines. In a networked deployment, the machine 700 can operate in the capacity of a server machine or a client machine in a server-client network environment, or as a peer machine in a distributed (e.g., peer-to-peer) network environment. The machine 700 can be a server computer, a client computer, a personal computer (PC), a tablet computer, a laptop computer, a netbook, a cellular telephone, a smartphone, a set-top box (STB), a personal digital assistant (PDA), a web appliance, a network router, a network switch, a network bridge, or any machine capable of executing the instructions 724, sequentially or otherwise, that specify actions to be taken by that machine. Further, while only a single machine is illustrated, the term “machine” shall also be taken to include any collection of machines that individually or jointly execute the instructions 724 to perform all or part of any one or more of the methodologies discussed herein.

The machine 700 includes a processor 702 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 704, and a static memory 706, which are configured to communicate with each other via a bus 708. The processor 702 can contain microcircuits that are configurable, temporarily, or permanently, by some or all of the instructions 724 such that the processor 702 is configurable to perform any one or more of the methodologies described herein, in whole or in part. For example, a set of one or more microcircuits of the processor 702 can be configurable to execute one or more modules (e.g., software modules) described herein.

The machine 700 can further include a graphics display 710 (e.g., a plasma display panel (PDP), a light emitting diode (LED) display, a liquid crystal display (LCD), a projector, a cathode ray tube (CRT), or any other display capable of displaying graphics or video). The machine 700 can also include an alphanumeric input device 712 (e.g., a keyboard or keypad), a cursor control device 714 (e.g., a mouse, a touchpad, a trackball, a joystick, a motion sensor, an eye tracking device, or other pointing instrument), a storage unit 716, an audio generation device 718 (e.g., a sound card, an amplifier, a speaker, a headphone jack, any suitable combination thereof, or any other suitable signal generation device), and a network interface device 720.

The storage unit 716 includes the machine-storage medium 722 (e.g., a tangible and non-transitory machine-storage medium) on which are stored the instructions 724, embodying any one or more of the methodologies or functions described herein. The instructions 724 can also reside, completely or at least partially, within the main memory 704, within the processor 702 (e.g., within the processor's cache memory), or both, before or during execution thereof by the machine 700. Accordingly, the main memory 704 and the processor 702 can be considered machine-storage media (e.g., tangible, and non-transitory machine-storage media). The instructions 724 can be transmitted or received over the network 726 via the network interface device 720. For example, the network interface device 720 can communicate the instructions 724 using any one or more transfer protocols (e.g., Hypertext Transfer Protocol (HTTP)).

In some example embodiments, the machine 700 can be a portable computing device, such as a smart phone or tablet computer, and have one or more additional input components (e.g., sensors 728 or gauges). Examples of the additional input components include an image input component (e.g., one or more cameras), an audio input component (e.g., a microphone), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components can be accessible and available for use by any of the modules described herein.

### Executable Instructions and Machine-Storage Medium

The various memories (i.e., 704, 706, and/or memory of the processor(s) 702) and/or storage unit 716 can store one or more sets of instructions and data structures (e.g., software) 724 embodying or utilized by any one or more of the methodologies or functions described herein. These instructions, when executed by processor(s) 702 cause various operations to implement the disclosed embodiments.

As used herein, the terms “machine-storage medium,” “device-storage medium,” “computer-storage medium” (referred to collectively as “machine-storage medium 722”) mean the same thing and can be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 722 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage medium or media, computer-storage medium or media, and device-storage medium or media 722 specifically exclude carrier waves, modulated data signals, and other such media, at least some of which are covered under the term “signal medium” discussed below. In this context, the machine-storage medium is non-transitory.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer Readable Medium

The terms “machine-readable medium,” “computer-readable medium” and “device-readable medium” mean the same thing and can be used interchangeably in this disclosure. The terms are defined to include both machine-storage media and signal media. Thus, the terms include both storage devices/media and carrier waves/modulated data signals.

The following, non-limiting examples, detail certain aspects of the present subject matter to solve the challenges and provide the benefits discussed herein, among others.

Aspect 1 is a method of automated cell culturing and characterization to resemble certain in Vivo environment conditions and promote desired cellular growth, the method comprising: regulating one or more parameters of an ambient environment within an environmentally isolated, airtight enclosure; culturing a first biological specimen variety, including: aspirating, via an automated fluid handling system having a fluidic interface disposed within the airtight enclosure, a portion of the first biological specimen variety; dispensing, via the fluidic interface of the automated fluid handling system, the portion of the first biological specimen variety in a first vessel, including suspending individual cells of the portion of the first biological specimen variety within a microcarrier matrix contained by the first vessel and exposed to the ambient environment within the airtight enclosure; monitoring at least one cellular growth indicator within the first vessel over time; and establishing or adjusting mechanical movement of the first vessel, based on the at least one cellular growth indicator, to promote growth of an ex Vivo cell culture within the first vessel; wherein the ambient environment within the airtight enclosure remains environmentally isolated from an outside environment during the culturing of the first biological specimen variety.

In Aspect 2, the subject matter of Aspect 1 includes, receiving a first biological specimen variety within the airtight enclosure.

In Aspect 3, the subject matter of Aspects 1-2 includes, wherein establishing or adjusting the mechanical movement of the first vessel includes: bidirectionally oscillating the first vessel via alternating a rotation of the vessel at least one hundred eighty degrees in each direction, including translating a rotational motion from the bidirectional oscillation to lateral, vertical motion of the microcarrier matrix contained by the first vessel.

In Aspect 4, the subject matter of Aspects 1-3 includes, wherein culturing the first biological specimen variety includes controlling movement, via a robotic manipulator, of the fluidic interface of the automated fluid handling system, toward the first vessel.

In Aspect 5, the subject matter of Aspect 4 includes, wherein controlling the movement of the fluidic interface includes placing a pipette tip of the fluidic interface within plus or minus three mm of a target location.

In Aspect 6, the subject matter of Aspects 1-5 includes, wherein culturing the first biological specimen variety includes: monitoring a pH value within the first vessel over a specified duration; determining a change in pH over the specified duration exceeding a threshold; and in response, triggering replacement of fluid within the first vessel, via the automated fluid handling system, upon the determination that change in pH exceeds the threshold.

In Aspect 7, the subject matter of Aspects 1-6 includes, wherein regulating the one or more parameters of the ambient environment within the airtight enclosure includes establishing or adjusting an ambient temperature to maintain a range from between fifty degrees Fahrenheit (F) to one hundred fifty degrees F.

In Aspect 8, the subject matter of Aspects 1-7 includes, wherein regulating the one or more parameters of the ambient environment within the airtight enclosure includes establishing or adjusting an ambient temperature to maintain a range from between ninety-three degrees Fahrenheit (F) to one hundred seven F.

In Aspect 9, the subject matter of Aspects 1-8 includes, wherein regulating the one or more parameters of the ambient environment within the airtight enclosure includes establishing or adjusting an ambient relative humidity (RH) to maintain a range from between seventy-five percent to one hundred percent.

In Aspect 10, the subject matter of Aspects 1-9 includes, wherein regulating the one or more parameters of the ambient environment within the airtight enclosure includes establishing or adjusting an ambient relative humidity (RH) to maintain a range from between forty-five percent to eighty percent.

In Aspect 11, the subject matter of Aspects 1-10 includes, wherein regulating the one or more parameters of the ambient environment within the airtight enclosure includes establishing or adjusting an ambient carbon dioxide concentration to maintain a range from between zero percent to fifteen percent.

In Aspect 12, the subject matter of Aspects 1-11 includes, wherein regulating the one or more parameters of the ambient environment within the airtight enclosure includes establishing or adjusting an ambient oxygen concentration to maintain a range from between five percent to twenty-five percent.

In Aspect 13, the subject matter of Aspects 1-12 includes, receiving a second biological specimen variety within the airtight enclosure; culturing the second biological specimen variety, including: aspirating, via the automated fluid handling system disposed within the airtight enclosure, a portion of the second biological specimen variety; dispensing, via the automated fluid handling system, the portion of the second biological specimen variety within a second vessel, including suspending individual cells of the portion of the second biological specimen variety within a microcarrier matrix contained by the second vessel and exposed to the ambient environment within the airtight enclosure; monitoring at least one cellular growth indicator within the second vessel over time; and establishing or adjusting an oscillation of the second vessel, based on the at least one cellular growth indicator, to promote growth of a cell culture within the second vessel.

In Aspect 14, the subject matter of Aspect 13 includes, at least one of washing or sterilizing a pipette tip of a fluidic interface included in the fluid handling system between the dispensing of the portion of the first biological specimen variety within the first vessel and the aspirating the portion of the second biological specimen variety; wherein the washing or sterilizing the pipette tip includes moving the fluidic interface, via a robotic manipulator, toward a washing or sterilization unit disposed within the environmentally isolated, airtight enclosure.

In Aspect 15, the subject matter of Aspects 1-14 includes, monitoring the at least one cellular growth indicator within the first vessel at a location outside the environmentally isolated, airtight enclosure.

In Aspect 16, the subject matter of Aspects 1-15 includes, disposing a ferromagnetic-infused biomimetic hydrogel microcarrier matrix within the first vessel, the hydrogel microcarrier matrix configured to receive the individual cells of the biological specimen and suspend the individual cells throughout the hydrogel microcarrier matrix during the culturing of the first biological specimen variety.

Aspect 17 is a system for automated cell culturing and characterization to resemble certain in Vivo environment conditions and promote desired cellular growth, the system comprising: an environmentally isolated, airtight enclosure for defining an ambient environment therewithin; an automated fluid handling system having a fluidic interface disposed within the airtight enclosure, the handling system configured to selectively: aspirate a portion of a first biological specimen variety; and dispense the portion of the first biological specimen variety in a first vessel; wherein the ambient environment within the airtight enclosure remains environmentally isolated from an outside environment during the selective aspiration and dispensing of the first biological specimen variety; and processing circuitry configured to: monitor at least one cellular growth indicator within the first vessel over time; and establish or adjust mechanical movement of the first vessel, based on the at least one cellular growth indicator, to promote growth of an ex Vivo cell culture within the first vessel.

In Aspect 18, the subject matter of Aspect 17 includes, a first vessel including a microcarrier matrix contained by the first vessel and exposed to the ambient environment within the airtight enclosure, the microcarrier matrix for receiving individual cells of the portion of the first biological specimen variety.

In Aspect 19, the subject matter of Aspect 18 includes, wherein the microcarrier matrix includes a ferromagnetic-infused biomimetic hydrogel microcarrier matrix configured to receive the individual cells of the biological specimen and suspend the individual cells throughout the hydrogel microcarrier matrix during the culturing of the first biological specimen variety.

In Aspect 20, the subject matter of Aspects 18-19 includes, a vessel manipulator configured to bidirectionally oscillate the first vessel via alternating a rotation of the vessel at least one hundred eighty degrees in each direction.

In Aspect 21, the subject matter of Aspect 20 includes, wherein, the first vessel is configured to translate a rotational motion from the bidirectional oscillation to lateral, vertical motion of the microcarrier matrix contained by the first vessel.

In Aspect 22, the subject matter of Aspects 17-21 includes, a robotic manipulator configured to control movement of the fluidic interface of the automated fluid handling system toward the first vessel.

In Aspect 23, the subject matter of Aspect 22 includes, wherein the robotic manipulator is configured to place a pipette tip of the fluidic interface within plus or minus zero point three mm of a target location.

In Aspect 24, the subject matter of Aspects 17-23 includes, a pH sensor for measuring a pH value within the first vessel over a specified duration and communicatively coupled with the processing circuitry; wherein the processing circuitry is configured to: determine a change in pH over the specified duration exceeding a threshold; and in response, trigger replacement of fluid within the first vessel, via the automated fluid handling system, upon the determination that change in pH exceeds the threshold.

In Aspect 25, the subject matter of Aspects 17-24 includes, a washing or sterilization unit disposed within the environmentally isolated, airtight enclosure; and a robotic manipulator configured to moving the fluidic interface toward the washing or sterilization unit to facilitate at least one of washing or sterilizing a pipette tip of a fluidic interface included in the fluid handling system.

Aspect 26 is at least one non-transitory machine-readable medium including instructions for facilitating automated cell culturing and characterization to resemble certain in Vivo environment conditions and promote desired cellular growth, which when executed by a processor, cause the processor to: regulate one or more parameters of an ambient environment within an environmentally isolated, airtight enclosure; and culture a first biological specimen variety, including: aspirate, via an automated fluid handling system having a fluidic interface disposed within the airtight enclosure, a portion of the first biological specimen variety; dispense, via the fluidic interface of the automated fluid handling system, the portion of the first biological specimen variety in a first vessel, including suspending individual cells of the portion of the first biological specimen variety within a microcarrier matrix contained by the first vessel and exposed to the ambient environment within the airtight enclosure; monitor at least one cellular growth indicator within the first vessel over time; and establish or adjust mechanical movement of the first vessel, based on the at least one cellular growth indicator, to promote growth of an ex Vivo cell culture within the first vessel; wherein the ambient environment within the airtight enclosure remains environmentally isolated from an outside environment during the culturing of the first biological specimen variety.

In Aspect 27, the subject matter of Aspect 26 includes, wherein establishing or adjusting the mechanical movement of the first vessel includes: bidirectionally oscillating the first vessel via alternating a rotation of the vessel at least one hundred eighty degrees in each direction, including translating a rotational motion from the bidirectional oscillation to lateral, vertical motion of the microcarrier matrix contained by the first vessel.

In Aspect 28, the subject matter of Aspects 26-27 includes, wherein culturing the first biological specimen variety includes controlling movement, via a robotic manipulator, of the fluidic interface of the automated fluid handling system, toward the first vessel.

In Aspect 29, the subject matter of Aspect 28 includes, wherein controlling the movement of the fluidic interface includes placing a pipette tip of the fluidic interface within plus or minus zero point three mm of a target location.

In Aspect 30, the subject matter of Aspects 26-29 includes, wherein culturing the first biological specimen variety includes: monitoring a pH value within the first vessel over a specified duration; determining a change in pH over the specified duration exceeding a threshold; and in response, triggering replacement of fluid within the first vessel, via the automated fluid handling system, upon the determination that change in pH exceeds the threshold.

In Aspect 31, the subject matter of Aspects 26-30 includes, instructions which cause the processor to: receive a second biological specimen variety within the airtight enclosure; and culture the second biological specimen variety, including: aspirate, via the automated fluid handling system disposed within the airtight enclosure, a portion of the second biological specimen variety; dispense, via the automated fluid handling system, the portion of the second biological specimen variety within a second vessel, including suspending individual cells of the portion of the second biological specimen variety within a microcarrier matrix contained by the second vessel and exposed to the ambient environment within the airtight enclosure; monitor at least one cellular growth indicator within the second vessel over time; and establish or adjust an oscillation of the second vessel, based on the at least one cellular growth indicator, to promote growth of a cell culture within the second vessel.

In Aspect 32, the subject matter of Aspect 31 includes, instructions which cause the processor to at least one of wash or sterilize a pipette tip of a fluidic interface included in the fluid handling system between the dispensing of the portion of the first biological specimen variety within the first vessel and the aspirating the portion of the second biological specimen variety; wherein the washing or sterilizing the pipette tip includes moving the fluidic interface, via a robotic manipulator, toward a washing or sterilization unit disposed within the environmentally isolated, airtight enclosure.

Aspect 33 is at least one machine-readable medium including instructions that, when executed by processing circuitry, cause the processing circuitry to perform operations to implement of any of Aspects 1-32.

Aspect 34 is an apparatus comprising means to implement of any of Aspects 1-32.

Aspect 35 is a system to implement of any of Aspects 1-32.

Aspect 36 is a method to implement of any of Aspects 1-32.

The above Detailed Description can include references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to help allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

What is claimed is:

1. A method of automated cell culturing and characterization to resemble certain in Vivo environment conditions and promote desired cellular growth, the method comprising:
regulating one or more parameters of an ambient environment within an environmentally isolated, airtight enclosure;
culturing a first biological specimen variety, including:
aspirating, via an automated fluid handling system having a fluidic interface disposed within the airtight enclosure, a portion of the first biological specimen variety;
dispensing, via the fluidic interface of the automated fluid handling system, the portion of the first biological specimen variety in a first vessel, including suspending individual cells of the portion of the first biological specimen variety within a microcarrier matrix contained by the first vessel and exposed to the ambient environment within the airtight enclosure;
monitoring at least one cellular growth indicator within the first vessel over time;
establishing or adjusting mechanical movement of the first vessel, based on the at least one cellular growth indicator, to promote growth of an ex Vivo cell culture within the first vessel;
wherein the ambient environment within the airtight enclosure remains environmentally isolated from an outside environment during the culturing of the first biological specimen variety.

2. The method of claim 1, comprising receiving a first biological specimen variety within the airtight enclosure.

3. The method of claim 1, wherein establishing or adjusting the mechanical movement of the first vessel includes:
bidirectionally oscillating the first vessel via alternating a rotation of the vessel at least 180° in each direction, including translating a rotational motion from the bidirectional oscillation to lateral, vertical motion of the microcarrier matrix contained by the first vessel.

4. The method of claim 1, wherein culturing the first biological specimen variety includes controlling movement, via a robotic manipulator, of the fluidic interface of the automated fluid handling system, toward the first vessel.

5. The method of claim 4, wherein controlling the movement of the fluid handler includes placing a pipette tip of the fluid handler within ±0.3 mm of a target location.

6. The method of claim 1, wherein culturing the first biological specimen variety includes:
monitoring a pH value within the first vessel over a specified duration;
determining a change in pH over the specified duration exceeding a threshold; and
in response, triggering replacement of fluid within the first vessel, via the automated fluid handling system, upon the determination that change in pH exceeds the threshold.

7. The method of claim 1, wherein regulating the one or more parameters of the ambient environment within the airtight enclosure includes establishing or adjusting an ambient temperature to maintain a range from between 50° Fahrenheit (F) to 150° F.

8. The method of claim 1, wherein regulating the one or more parameters of the ambient environment within the airtight enclosure includes establishing or adjusting an ambient temperature to maintain a range from between 93° Fahrenheit (F) to 107° F.

9. The method of claim 1, wherein regulating the one or more parameters of the ambient environment within the airtight enclosure includes establishing or adjusting an ambient relative humidity (RH) to maintain a range from between 75%-100%.

10. The method of claim 1, wherein regulating the one or more parameters of the ambient environment within the airtight enclosure includes establishing or adjusting an ambient relative humidity (RH) to maintain a range from between 45%-80%.

11. The method of claim 1, wherein regulating the one or more parameters of the ambient environment within the airtight enclosure includes establishing or adjusting an ambient CO² concentration to maintain a range from between 0%-15%.

12. The method of claim 1, wherein regulating the one or more parameters of the ambient environment within the airtight enclosure includes establishing or adjusting an ambient O² concentration to maintain a range from between 5%-25%.

13. The method of claim 1, comprising:
receiving a second biological specimen variety within the airtight enclosure;
culturing the second biological specimen variety, including:
aspirating, via the automated fluid handling system disposed within the airtight enclosure, a portion of the second biological specimen variety;
dispensing, via the automated fluid handling system, the portion of the second biological specimen variety within a second vessel, including suspending individual cells of the portion of the second biological specimen variety within a microcarrier matrix contained by the second vessel and exposed to the ambient environment within the airtight enclosure;
monitoring at least one cellular growth indicator within the second vessel over time;
establishing or adjusting an oscillation of the second vessel, based on the at least one cellular growth indicator, to promote growth of a cell culture within the second vessel.

14. The method of claim 13, comprising at least one of washing or sterilizing a pipette tip of a fluid handler included in the fluid handling system between the dispensing of the portion of the first biological specimen variety within the first vessel and the aspirating the portion of the second biological specimen variety;
wherein the washing or sterilizing the pipette tip includes moving the fluid handler, via a robotic manipulator, toward a washing or sterilization unit disposed within the sealed, airtight enclosure.

15. The method of claim 1, comprising monitoring the at least one cellular growth indicator within the first vessel at a location outside the sealed, airtight enclosure.

16. The method of claim 1, comprising disposing a ferromagnetic-infused biomimetic hydrogel microcarrier matrix within the first vessel, the hydrogel microcarrier matrix configured to receive the individual cells of the biological specimen and suspend the individual cells throughout the hydrogel microcarrier matrix during the culturing of the first biological specimen variety.

17. A system for automated cell culturing and characterization to resemble certain in Vivo environment conditions and promote desired cellular growth, the system comprising:
an environmentally isolated, airtight enclosure for defining an ambient environment therewithin;
an automated fluid handling system having a fluidic interface disposed within the airtight enclosure, the handling system configured to selectively:
aspirate a portion of a first biological specimen variety; and
dispense the portion of the first biological specimen variety in a first vessel;
wherein the ambient environment within the airtight enclosure remains environmentally isolated from an outside environment during the selective aspiration and dispensing of the first biological specimen variety; and
processing circuitry configured to:
monitor at least one cellular growth indicator within the first vessel over time; and
establish or adjust mechanical movement of the first vessel, based on the at least one cellular growth indicator, to promote growth of an ex Vivo cell culture within the first vessel.

18. The system of claim 17, comprising:
a first vessel including a microcarrier matrix contained by the first vessel and exposed to the ambient environment within the airtight enclosure, the microcarrier matrix for receiving individual cells of the portion of the first biological specimen variety.

19. The system of claim 18, wherein the microcarrier matrix includes a ferromagnetic-infused biomimetic hydrogel microcarrier matrix configured to receive the individual cells of the biological specimen and suspend the individual cells throughout the hydrogel microcarrier matrix during the culturing of the first biological specimen variety.

20. The system of claim 18, comprising a vessel manipulator configured to bidirectionally oscillate the first vessel via alternating a rotation of the vessel at least 180° in each direction.

21. The system of claim 20, wherein, the first vessel is configured to translate a rotational motion from the bidirectional oscillation to lateral, vertical motion of the microcarrier matrix contained by the first vessel.

22. The system of claim 17, comprising a robotic manipulator configured to control movement of the fluidic interface of the automated fluid handling system toward the first vessel.

23. The system of claim 22, wherein the robotic manipulator is configured to place a pipette tip of the fluidic interface within ±0.3 mm of a target location.

24. The system of claim 17, comprising:
a pH sensor for measuring a pH value within the first vessel over a specified duration and communicatively coupled with the processing circuitry;
wherein the processing circuitry is configured to:
determine a change in pH over the specified duration exceeding a threshold; and
in response, trigger replacement of fluid within the first vessel, via the automated fluid handling system, upon the determination that change in pH exceeds the threshold.

25. The system of claim 17, comprising:
a washing or sterilization unit disposed within the environmentally isolated, airtight enclosure; and
a robotic manipulator configured to moving the fluidic interface toward the washing or sterilization unit to facilitate at least one of washing or sterilizing a pipette tip of a fluidic interface included in the fluid handling system.

26. At least one non-transitory machine-readable medium including instructions for facilitating automated cell culturing and characterization to resemble certain in Vivo environment conditions and promote desired cellular growth, which when executed by a processor, cause the processor to:
regulate one or more parameters of an ambient environment within an environmentally isolated, airtight enclosure; and
culture a first biological specimen variety, including:
aspirate, via an automated fluid handling system having a fluidic interface disposed within the airtight enclosure, a portion of the first biological specimen variety;
dispense, via the fluidic interface of the automated fluid handling system, the portion of the first biological specimen variety in a first vessel, including suspending individual cells of the portion of the first biological specimen variety within a microcarrier matrix contained by the first vessel and exposed to the ambient environment within the airtight enclosure;
monitor at least one cellular growth indicator within the first vessel over time; and
establish or adjust mechanical movement of the first vessel, based on the at least one cellular growth indicator, to promote growth of an ex Vivo cell culture within the first vessel;
wherein the ambient environment within the airtight enclosure remains environmentally isolated from an outside environment during the culturing of the first biological specimen variety.

27. The at least one non-transitory machine-readable medium of claim 26, wherein establishing or adjusting the mechanical movement of the first vessel includes:
bidirectionally oscillating the first vessel via alternating a rotation of the vessel at least 180° in each direction, including translating a rotational motion from the bidirectional oscillation to lateral, vertical motion of the microcarrier matrix contained by the first vessel.

28. The at least one non-transitory machine-readable medium of claim 26, wherein culturing the first biological specimen variety includes controlling movement, via a robotic manipulator, of the fluidic interface of the automated fluid handling system, toward the first vessel.

29. The at least one non-transitory machine-readable medium of claim 28, wherein controlling the movement of the fluidic interface includes placing a pipette tip of the fluidic interface within ±0.3 mm of a target location.

30. The at least one non-transitory machine-readable medium of claim 26, wherein culturing the first biological specimen variety includes:
monitoring a pH value within the first vessel over a specified duration;
determining a change in pH over the specified duration exceeding a threshold; and
in response, triggering replacement of fluid within the first vessel, via the automated fluid handling system, upon the determination that change in pH exceeds the threshold.

31. The at least one non-transitory machine-readable medium of claim 26, including instructions which cause the processor to:
identify a second biological specimen variety within the airtight enclosure; and
culture the second biological specimen variety, including:
aspirate, via the automated fluid handling system disposed within the airtight enclosure, a portion of the second biological specimen variety;
dispense, via the automated fluid handling system, the portion of the second biological specimen variety within a second vessel, including suspending individual cells of the portion of the second biological specimen variety within a microcarrier matrix contained by the second vessel and exposed to the ambient environment within the airtight enclosure;
monitor at least one cellular growth indicator within the second vessel over time; and
establish or adjust an oscillation of the second vessel, based on the at least one cellular growth indicator, to promote growth of a cell culture within the second vessel.

32. The at least one non-transitory machine-readable medium of claim 31, including instructions which cause the processor to at least one of wash or sterilize a pipette tip of a fluidic interface included in the fluid handling system between the dispensing of the portion of the first biological specimen variety within the first vessel and the aspirating the portion of the second biological specimen variety;
wherein the washing or sterilizing the pipette tip includes moving the fluidic interface, via a robotic manipulator, toward a washing or sterilization unit disposed within the environmentally isolated, airtight enclosure.
